(19) United States
(12) Patent Application Publication
(10) Pub. No.: US 2024/0335091 A1
(43) Pub. Date: Oct. 10, 2024

(54) SYSTEMS AND METHODS FOR PROVIDING MEDICAL FLUORESCENCE IMAGING WITH A MODULATED FLUORESCENCE EXCITATION ILLUMINATION SOURCE

(71) Applicant: Stryker Corporation, Kalamazoo, MI (US)

(72) Inventors: Ajay RAMESH, Pleasanton, CA (US); William Huei Liang CHANG, Milpitas, CA (US)

(73) Assignee: Stryker Corporation, Kalamazoo, MI (US)

(21) Appl. No.: 18/628,640

(22) Filed: Apr. 5, 2024

Related U.S. Application Data
(60) Provisional application No. 63/494,989, filed on Apr. 7, 2023.

(51) Int. Cl. A61B 1/00; A61B 1/04; A61B 1/045; A61B 1/05; A61B 1/06; A61B 5/00

(52) U.S. Cl. CPC A61B 1/000095; A61B 1/00117; A61B 1/043; A61B 1/045; A61B 1/046; A61B 1/05; A61B 1/0638; A61B 1/0655; A61B 1/0669; A61B 1/0684; A61B 5/0071

(57) ABSTRACT

The present disclosure relates generally to medical imaging, and more specifically to techniques for providing medical fluorescence imaging with modulated illumination source. An exemplary method comprises illuminating a tissue of a subject with a white-light illumination source for a first illumination period, wherein the fluorescence excitation illumination source is off during the first illumination period; producing a first set of imaging data; illuminating the tissue of the subject with a fluorescence excitation illumination source for a second illumination period after the first illumination period, wherein the white-light illumination source is off during the second illumination period; producing a second set of imaging data; and generating one or more image frames based on the first set of imaging data and the second set of imaging data.

### CROSS-REFERENCE TO RELATED APPLICATIONS

This application claims the benefit of U.S. Provisional Application 63/494,989, filed Apr. 7, 2023, the entire content of which is incorporated herein by reference for all purposes.

### FIELD

The present disclosure relates generally to medical imaging, and more specifically to techniques for providing medical fluorescence imaging with a modulated fluorescence excitation illumination source.

### BACKGROUND

Medical systems, instruments or tools are utilized pre-surgery, during surgery, or post-operatively for various purposes. In particular, medical imaging systems can be used to enable a surgeon to view a surgical site in open-field procedures and endoscopic procedures. For example, endoscopy in the medical field allows internal features of the body of a patient to be viewed without the use of traditional, fully invasive surgery. Endoscopic imaging systems incorporate endoscopes to enable a surgeon to view a surgical site, and endoscopic tools enable non-invasive surgery at the site. Endoscopes may be usable along with a camera system for processing the images received by the endoscope. An endoscopic camera system typically includes a camera head connected to a camera control unit (CCU) that processes input image data received from the image sensor of the camera and outputs the image data for display. The CCU may control an illuminator that generates illumination light provided to the imaged scene.

Various imager sensors may be used in imaging systems (e.g., endoscopic imaging systems, open-field imaging systems), including charge-coupled device (CCD) sensors and complementary metal oxide semiconductor (CMOS) sensors. The construction of CCDs is generally more complex than the construction of CMOS sensors, and CMOS sensors may be built in high volume wafer fabrication facilities used for related technologies such as microprocessors and chip sets. As a result, CMOS sensors are often less costly than CCDs for similar performance. In addition to lower cost, the common fabrication processes used to create CMOS imagers permits a CMOS pixel array to be integrated on a single circuit with other electronic devices such as clock drivers, digital logic, analog/digital converters, and other suitable electronics. The compact structures possible for a CMOS imager may also reduce space requirements and lower power consumption. CMOS imagers can also have higher sensitivity and provide higher video frame rates.

CMOS based imagers may utilize electronic rolling shutters to expose pixels in the sensor array. With an electronic rolling shutter, rows of pixels are cleared (or reset), exposed, and read out in sequence. During integration, a row of pixels is exposed to light energy and each pixel builds an electric charge corresponding to the amount and wavelengths of light impinging on the pixel. Because the rows are activated and read out in sequence, there is an elapsed time between when the first row integrates and when the last row integrates. Because of the elapsed time between when the first row begins to integrate and when the subsequent rows begin to integrate, a CMOS imager with an electronic rolling shutter may capture video images with blur or other rolling shutter effects. CMOS based imagers may also utilize global shutters to expose pixels in the sensor array. With a global shutter, all rows of pixels are exposed at the same time (i.e., same start and end of exposure) but the readout may be (and usually is) sequential.

Medical imaging systems (e.g., endoscopic imaging systems, open-field imaging systems) may include both a white-light illumination source and a fluorescence excitation illumination source. The white-light illumination source and the fluorescence excitation illumination source can be used to illuminate a tissue of a subject to obtain different types of image data (e.g., fluorescence image frames, white-light image frames, blended image frames). In particular, the fluorescence excitation illumination source, such as an infrared light or a blue-light illumination source, can provide illumination to the imaged tissue to excite the fluorophores (or fluorochromes) to produce fluorescence emission. In many medical imaging systems, the fluorescence excitation illumination source is configured to be constantly on during an imaging session in order to provide maximum exposure time for the fluorescence image frames and to provide maximum fluorescence excitation strength. However, this always-on configuration has a number of deficiencies. For example, when the imaged tissue is close to the surgical imaging device that is emitting the illumination (e.g., at the minimum working distance), the fluorescence excitation strength can be excessively high and cause an intense fluorescence emission that in turn generates a strong fluorescence signal in the camera of the imaging device. The strong fluorescence signal generated by the emission may reduce the background/white-light average luminance of an image frame that has a fluorescence component and contaminate the image frame. For example, if the fluorescence signal is represented in green, the entire scene in the image frame may appear green, thus creating an illusion of false fluorescence signals in the camera of the imaging device. This can be problematic for open-field imaging systems and endoscopic imaging systems. For example, the endoscopic cameras can get very close to the tissue and can also move around the tissue quickly during surgeries, thus generating false fluorescence signals that can compromise surgical safety.

### SUMMARY

Examples of the present disclosure include techniques for modulating a fluorescence excitation illumination source of an imaging system to improve the stability and consistency of the fluorescence signals. According to some examples of a modulation scheme, the fluorescence excitation illumination source and the white-light illumination source are configured to alternately provide illumination periods to the tissue of the subject. According to some examples of the modulation scheme, the fluorescence excitation illumination source is configured to provide illumination periods to the tissue of the subject at a frequency, and the white-light illumination source is configured to provide illumination during at least part of every other illumination period of the illumination periods of the fluorescence excitation illumination source; further, the length of each illumination period of the fluorescence excitation illumination source can be dynamically adjusted based on the distance between the imaging device conveying the illumination and the tissue. Both modulation schemes provide a number of technical advantages over the always-on fluorescence illumination scheme, as described below.

While some of the techniques are described with respect to a certain type of imager (e.g., a rolling shutter imager, a global shutter imager), it should be appreciated that the techniques can be applied in any type of imager. Further, the techniques can be applied in non-surgical or non-medical uses.

An exemplary method of imaging tissue of a subject using an imaging system comprising a rolling shutter imager, a fluorescence excitation illumination source, and a white-light illumination source, comprises: illuminating the tissue of the subject with the white-light illumination source for a first illumination period to accumulate charge at a plurality of rows of pixels of the rolling shutter imager, wherein the fluorescence excitation illumination source is off during the first illumination period; sequentially reading a first set of accumulated charge at the plurality of rows of pixels from a first row to a last row of the plurality of rows to produce a first set of imaging data; illuminating the tissue of the subject with the fluorescence excitation illumination source for a second illumination period after the first illumination period to accumulate charge at the plurality of rows of pixels of the rolling shutter imager, wherein the white-light illumination source is off during the second illumination period; sequentially reading a second set of accumulated charge at the plurality of rows of pixels from the first row to the last row of the plurality of rows to produce a second set of imaging data; and generating one or more image frames based on the first set of imaging data and the second set of imaging data.

In some examples, the fluorescence excitation illumination source and the white-light illumination source are configured to alternately provide illumination periods to the tissue of the subject.

In some examples, the fluorescence excitation illumination source is configured to provide illumination periods at a first frequency and the white-light illumination source is configured to provide illumination periods at a second frequency.

In some examples, the first frequency or the second frequency corresponds to the frame rate of the rolling shutter imager.

In some examples, the first frequency or the second frequency is half the frame rate of the rolling shutter imager.

In some examples, each illumination period of the white-light illumination source is a variable configured to not exceed a first maximum value.

In some examples, the first maximum value is about 1 millisecond.

In some examples, each illumination period of the fluorescence excitation illumination source is a fixed value.

In some examples, each illumination period of the fluorescence excitation illumination source is a variable configured to not exceed a second maximum value.

In some examples, the one or more image frames comprise a white-light image frame based on the first set of imaging data.

In some examples, the one or more image frames comprise a fluorescence image frame based on the second set of imaging data.

In some examples, the one or more image frames comprise a blended image frame based on the fluorescence image frame and the white-light image frame.

In some examples, fluorescence image frame is overlaid on the white-light image frame in the blended image frame.

In some examples, the blended image frame is derived from colorizing the white-light image frame based on the fluorescence image frame.

In some examples, the method further comprises: adding the one or more image frames to a video stream.

In some examples, the white-light illumination source comprises an LED.

In some examples, the fluorescence excitation illumination source comprises an infrared light, a blue-light illumination source, or any combination thereof.

In some examples, the rolling shutter imager is part of an endoscopic imager. The endoscopic imager can be pre-inserted prior to start of the imaging method.

In some examples, the rolling shutter imager comprises a CMOS sensor.

An exemplary method of imaging tissue of a subject using an imaging system comprising a rolling shutter imager, a fluorescence excitation illumination source, and a white-light illumination source, comprises: illuminating the tissue of the subject with the white-light illumination source for a first illumination period and with the fluorescence excitation illumination source for a second illumination period to accumulate charge at a plurality of rows of pixels of the rolling shutter imager; sequentially reading a first set of accumulated charge at the plurality of rows of pixels from a first row to a last row of the plurality of rows to produce a first set of imaging data; illuminating the tissue of the subject with the fluorescence excitation illumination source for a third illumination period after the second illumination period to accumulate charge at the plurality of rows of pixels of the rolling shutter imager, wherein the white-light illumination source is off during the second illumination period; sequentially reading a second set of accumulated charge at the plurality of rows of pixels from the first row to the last row of the plurality of rows to produce a second set of imaging data; and generating one or more image frames based on the first set of imaging data and the second set of imaging data.

In some examples, the fluorescence excitation illumination source is configured to provide illumination periods to the tissue of the subject at a first frequency, and the white-light illumination source is configured to provide illumination during at least part of every other illumination period of the illumination periods of the fluorescence excitation illumination source.

In some examples, the first frequency corresponds to the frame rate of the rolling shutter imager.

In some examples, the first frequency equals the frame rate of the rolling shutter imager.

In some examples, each illumination period of the white-light illumination source is a variable configured to not exceed a first maximum value.

In some examples, the first maximum value is about 1 millisecond.

In some examples, each illumination period of the fluorescence excitation illumination source is a variable based on a distance between an imaging device conveying the illumination and the tissue.

In some examples, the distance is determined based on a luminance value calculated based on the first set of imaging data.

In some examples, the one or more image frames comprise a white-light image frame obtained based on the first set of imaging data.

In some examples, the one or more image frames comprise a fluorescence frame obtained based on the second set of imaging data.

In some examples, the one or more image frames comprise a blended image frame of the fluorescence image frame and the white-light image frame.

In some examples, the fluorescence image frame is overlaid on the white-light image frame in the blended image frame.

In some examples, the blended image frame is derived from colorizing the white-light image frame based on the fluorescence image frame.

In some examples, the method further comprises: adding the one or more image frames to a video stream.

In some examples, the white-light illumination source comprises an LED.

In some examples, the fluorescence excitation illumination source comprises an infrared light, a blue-light illumination source, or any combination thereof.

In some examples, the rolling shutter imager is part of an endoscopic imager. The endoscopic imager can be pre-inserted prior to start of the imaging method.

In some examples, the rolling shutter imager comprises a CMOS sensor.

In some examples, the first illumination period and the second illumination period start at the same time.

In some examples, the second illumination period is longer than the first illumination period.

An exemplary method for enhancing a fluorescence medical image comprises: receiving a white-light medical image corresponding to the fluorescence medical image; and enhancing the fluorescence medical image by: determining, for each pixel in the white-light medical image, a maximum value among a plurality of color components of the pixel; and dividing a corresponding pixel in the fluorescence medical image by the maximum value. The method may include obtaining the white-light medical image.

In some examples, the method further comprises: displaying the enhanced fluorescence medical image.

In some examples, the enhanced fluorescence medical image is displayed according to a color scale in which different colors represent different fluorescence intensities.

In some examples, according to the color scale, red indicates a higher fluorescence intensity than green and green indicates a higher fluorescence intensity than blue.

In some examples, the enhanced fluorescence medical image is displayed as an overlay on the corresponding white-light image.

In some examples, displaying the enhanced fluorescence medical image comprises colorizing the corresponding white-light image based on the enhanced medical image and displaying the colorized white-light image.

In some examples, the plurality of color components comprises: a red component, a green component, a blue component, or any combination thereof.

In some examples, the fluorescence medical image and the white-light medical image depict the same tissue of a subject.

In some examples, the tissue comprises a lymph node of the subject.

In some examples, the fluorescence medical image and the white-light medical image are obtained using a rolling shutter imager.

In some examples, the fluorescence medical image and the white-light medical image are obtained using a global shutter imager.

In some examples, the fluorescence medical image and the white-light medical image are obtained using an endoscopic imager. The endoscopic imager can be pre-inserted prior to start of the enhancing method.

In some examples, the fluorescence medical image and the white-light medical image are obtained using an open-field imager.

An exemplary system of imaging tissue of a subject comprises: a fluorescence excitation illumination source, a white-light illumination source, an imaging apparatus that comprises an electronic rolling shutter. The imaging apparatus can be configured for: illuminating the tissue of the subject with the white-light illumination source for a first illumination period to accumulate charge at a plurality of rows of pixels of the rolling shutter imager, wherein the fluorescence excitation illumination source is off during the first illumination period; sequentially reading a first set of accumulated charge at the plurality of rows of pixels from a first row to a last row of the plurality of rows to produce a first set of imaging data; illuminating the tissue of the subject with the fluorescence excitation illumination source for a second illumination period after the first illumination period to accumulate charge at the plurality of rows of pixels of the rolling shutter imager, wherein the white-light illumination source is off during the second illumination period; sequentially reading a second set of accumulated charge at the plurality of rows of pixels from the first row to the last row of the plurality of rows to produce a second set of imaging data; and generating one or more image frames based on the first set of imaging data and the second set of imaging data.

In some examples, the fluorescence excitation illumination source and the white-light illumination source are configured to alternately provide illumination periods to the tissue of the subject.

In some examples, the fluorescence excitation illumination source is configured to provide illumination periods at a first frequency and the white-light illumination source is configured to provide illumination periods at a second frequency.

In some examples, the first frequency or the second frequency corresponds to the frame rate of the rolling shutter imager.

In some examples, the first frequency or the second frequency is half the frame rate of the rolling shutter imager.

In some examples, each illumination period of the white-light illumination source is a variable configured to not exceed a first maximum value.

In some examples, the first maximum value is about 1 millisecond.

In some examples, each illumination period of the fluorescence excitation illumination source is a fixed value.

In some examples, each illumination period of the fluorescence excitation illumination source is a variable configured to not exceed a second maximum value.

In some examples, the one or more image frames comprise a white-light image frame based on the first set of imaging data.

In some examples, the one or more image frames comprise a fluorescence image frame based on the second set of imaging data.

In some examples, the one or more image frames comprise a blended image frame based on the fluorescence image frame and the white-light image frame.

In some examples, the fluorescence image frame is overlaid on the white-light image frame in the blended image frame.

In some examples, the blended image frame is derived from colorizing the white-light image frame based on the fluorescence image frame.

In some examples, the imaging apparatus is further configured for: adding the one or more image frames to a video stream.

In some examples, the white-light illumination source comprises an LED.

In some examples, the fluorescence excitation illumination source comprises an infrared light, a blue-light illumination source, or any combination thereof.

In some examples, the rolling shutter imager is part of an endoscopic imager.

In some examples, the rolling shutter imager comprises a CMOS sensor.

An exemplary system of imaging tissue of a subject comprises: a fluorescence excitation illumination source, a white-light illumination source, and an imaging apparatus that comprises an electronic rolling shutter. The imaging apparatus can be configured for: illuminating the tissue of the subject with the white-light illumination source for a first illumination period and with the fluorescence excitation illumination source for a second illumination period to accumulate charge at a plurality of rows of pixels of the rolling shutter imager; sequentially reading a first set of accumulated charge at the plurality of rows of pixels from a first row to a last row of the plurality of rows to produce a first set of imaging data; illuminating the tissue of the subject with the fluorescence excitation illumination source for a third illumination period after the second illumination period to accumulate charge at the plurality of rows of pixels of the rolling shutter imager, wherein the white-light illumination source is off during the second illumination period; sequentially reading a second set of accumulated charge at the plurality of rows of pixels from the first row to the last row of the plurality of rows to produce a second set of imaging data; and generating one or more image frames based on the first set of imaging data and the second set of imaging data.

In some examples, the fluorescence excitation illumination source is configured to provide illumination periods to the tissue of the subject at a first frequency, and the white-light illumination source is configured to provide illumination during at least part of every other illumination period of the illumination periods of the fluorescence excitation illumination source.

In some examples, the first frequency corresponds to the frame rate of the rolling shutter imager.

In some examples, the first frequency equals the frame rate of the rolling shutter imager.

In some examples, each illumination period of the white-light illumination source is a variable configured to not exceed a first maximum value.

In some examples, the first maximum value is about 1 millisecond.

In some examples, each illumination period of the fluorescence excitation illumination source is a variable based on a distance between an imaging apparatus conveying the illumination and the tissue.

In some examples, the distance is determined based on a luminance value calculated based on the first set of imaging data.

In some examples, the one or more image frames comprise a white-light image frame obtained based on the first set of imaging data.

In some examples, the one or more image frames comprise a fluorescence frame obtained based on the second set of imaging data.

In some examples, the one or more image frames comprise a blended image frame of the fluorescence image frame and the white-light image frame.

In some examples, the fluorescence image frame is overlaid on the white-light image frame in the blended image frame.

In some examples, the blended image frame is derived from colorizing the white-light image frame based on the fluorescence image frame.

In some examples, the imaging apparatus is further configured for: adding the one or more image frames to a video stream.

In some examples, the white-light illumination source comprises an LED.

In some examples, the fluorescence excitation illumination source comprises an infrared light, a blue-light illumination source, or any combination thereof.

In some examples, the rolling shutter imager is part of an endoscopic imager.

In some examples, the rolling shutter imager comprises a CMOS sensor.

In some examples, the first illumination period and the second illumination period start at the same time.

In some examples, the second illumination period is longer than the first illumination period.

An exemplary system for enhancing a fluorescence medical image comprises: one or more processors; one or more memories; and one or more programs. The one or more programs are stored in the one or more memories and configured to be executed by the one or more processors.

The one or more programs include instructions for: obtaining a white-light medical image corresponding to the fluorescence medical image; and enhancing the fluorescence medical image by: determining, for each pixel in the white-light medical image, a maximum value among a plurality of color components of the pixel; and dividing a corresponding pixel in the fluorescence medical image by the maximum value.

In some examples, the one or more programs further include instructions for: displaying the enhanced fluorescence medical image.

In some examples, the enhanced fluorescence medical image is displayed according to a color scale in which different colors represent different fluorescence intensities.

In some examples, according to the color scale, red indicates a higher fluorescence intensity than green and green indicates a higher fluorescence intensity than blue.

In some examples, the enhanced fluorescence medical image is displayed as an overlay on the corresponding white-light image.

In some examples, displaying the enhanced fluorescence medical image comprises colorizing the corresponding white-light image based on the enhanced medical image and displaying the colorized white-light image.

In some examples, the plurality of color components comprises: a red component, a green component, a blue component, or any combination thereof.

In some examples, the fluorescence medical image and the white-light medical image depict the same tissue of a subject.

In some examples, the tissue comprises a lymph node of the subject.

In some examples, the fluorescence medical image and the white-light medical image are obtained using a rolling shutter imager.

In some examples, the fluorescence medical image and the white-light medical image are obtained using a global shutter imager.

In some examples, the fluorescence medical image and the white-light medical image are obtained using an endoscopic imager.

In some examples, the fluorescence medical image and the white-light medical image are obtained using an open-field imager.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be described, by way of example only, with reference to the accompanying drawings, in which:

FIG. 1A is an illustration of an endoscopic camera system, according to some examples;

FIG. 1B is a diagram of a portion of the endoscopic camera system of FIG. 1A and a target object for imaging, according to some examples;

FIG. 2 illustrates a schematic view of a system for illumination and imaging in open-field surgeries, according to some examples;

FIG. 3 is a block diagram of an imaging system, according to some examples;

FIGS. 4A and 4B illustrate an imaging system in which the fluorescence excitation illumination source is set to be always on without the option to modulate the source, according to some examples;

FIG. 5 provides an exemplary method for imaging tissue of a subject, in accordance with some examples;

FIG. 6A illustrates exemplary operations of an exemplary imaging system, in accordance with some examples;

FIG. 6B illustrates a timing diagram of the exemplary imaging system, in accordance with some examples;

FIG. 7 provides an exemplary method for imaging tissue of a subject, in accordance with some examples;

FIG. 8A illustrates exemplary operations of an exemplary imaging system, in accordance with some examples;

FIG. 8B illustrates a timing diagram of the exemplary imaging system, in accordance with some examples;

FIG. 9 illustrates an exemplary method for enhancing a fluorescence medical image, according to some examples;

FIG. 10 illustrates a comparison between three segmented images, in accordance with some examples;

FIG. 11 illustrates an exemplary blue-light fluorescence imaging system, in accordance with some examples.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described. Examples will now be described more fully hereinafter with reference to the accompanying drawings; however, they may be embodied in different forms and should not be construed as limited to the examples set forth herein. Rather, these examples are provided so that this disclosure will be thorough and complete, and will fully convey exemplary implementations to those skilled in the art.

Examples of the present disclosure include techniques for modulating a fluorescence excitation illumination source of an imaging system to improve the stability and consistency of the fluorescence signals. According to some examples of a modulation scheme, the fluorescence excitation illumination source and the white-light illumination source are configured to alternately provide illumination periods to the tissue of the subject. According to some examples of the modulation scheme, the fluorescence excitation illumination source is configured to provide illumination periods to the tissue of the subject at a frequency, and the white-light illumination source is configured to provide illumination during at least part of every other illumination period of the illumination periods of the fluorescence excitation illumination source; further, the length of each illumination period of the fluorescence excitation illumination source can be dynamically adjusted based on the distance between the imaging device conveying the illumination and the tissue. Both modulation schemes provide a number of technical advantages over the always-on fluorescence illumination scheme, as described below.

According to some examples of a modulation scheme, an exemplary system illuminates a tissue of a subject with a white-light illumination source for a first illumination period to accumulate charge at a plurality of rows of pixels of the rolling shutter imager. The fluorescence excitation illumination source is off during the first illumination period. The system sequentially reads a first set of accumulated charge at the plurality of rows of pixels from a first row to a last row of the plurality of rows to produce a first set of imaging data. The system then illuminates the tissue of the subject with the fluorescence excitation illumination source for a second illumination period after the first illumination period to accumulate charge at the plurality of rows of pixels of the rolling shutter imager. The white-light illumination source is off during the second illumination period. The system sequentially reads a second set of accumulated charge at the plurality of rows of pixels from the first row to the last row of the plurality of rows to produce a second set of imaging data. Based on the first set of imaging data and the second set of imaging data, the system generates one or more image frames based on the first set of imaging data and the second set of imaging data. In one particular implementation, the fluorescence excitation illumination source is modulated to be on for about 1 millisecond at about 60 Hz 1800 phase offset to the white-light illumination source.

The imaging system utilizing the above-described examples of modulation scheme may provide many technical advantages over imaging systems with the always-on fluorescence illumination excitation source. By isolating the white-light illumination and the fluorescence excitation illumination, the imaging system can reduce cross-contamination and gain better isolation between frames and the resulting blended image can be clearer. The imaging system can provide better control and consistency of the fluorescence images. For example, the imaging system can provide improved excitation illumination strength at the minimum working distance. However, the imaging system may have weaker performance as the surgical imaging device that is emitting the illumination gets farther away from the tissue. Because the fluorescence excitation illumination periods are set to not exceed a fixed value (e.g., only about one millisecond), if the imaging device conveying the illumination gets far enough from the tissue, the imaging system would lose the fluorescence strength due to the distance and the fixed exposure time.

According to some examples of a modulation scheme, an exemplary system illuminates the tissue of the subject with the white-light illumination source for a first illumination period and the fluorescence excitation illumination source for a second illumination period to accumulate charge at a plurality of rows of pixels of the rolling shutter imager. The first illumination period and the second illumination period may start at the same time and the second illumination period may be longer than the first illumination period. The system sequentially reads a first set of accumulated charge at the plurality of rows of pixels from a first row to a last row of the plurality of rows to produce a first set of imaging data. The system illuminates the tissue of the subject with the fluorescence excitation illumination source for a third illumination period after the second illumination period to accumulate charge at the plurality of rows of pixels of the rolling shutter imager. The white-light illumination source is off during the third illumination period. The system sequentially reads a second set of accumulated charge at the plurality of rows of pixels from the first row to the last row of the plurality of rows to produce a second set of imaging data. Based on the first set of imaging data and the second set of imaging data, the system generates one or more image frames based on the first set of imaging data and the second set of imaging data. In one particular implementation, the fluorescence excitation illumination source is modulated for dynamically varying pulse width between 10 microseconds-8.342 milliseconds at 120 Hz based on distance to the imaged tissue.

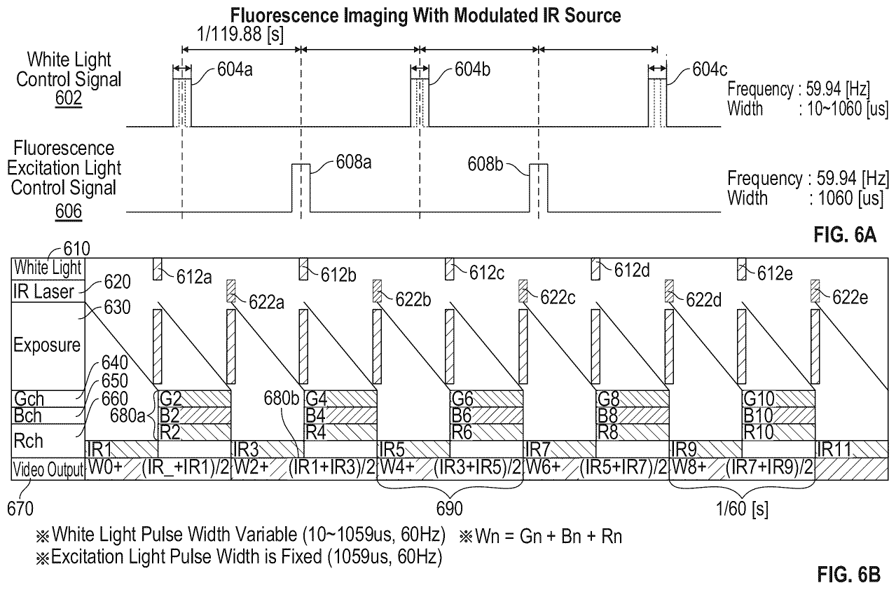

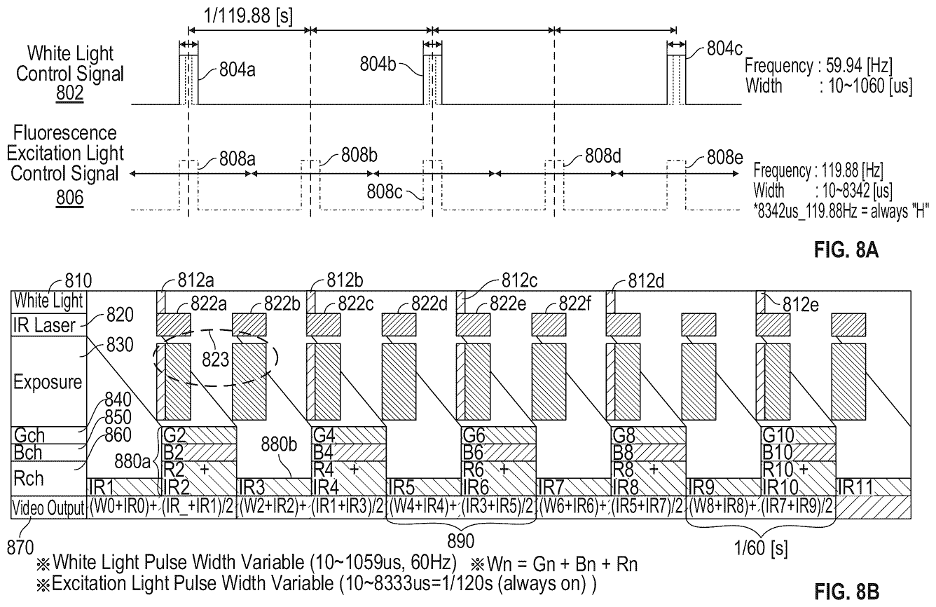

The imaging system utilizing the above-described examples of modulation scheme provides a number of technical advantages. First, the length of each illumination period of the fluorescence excitation illumination source is dynamically adjusted based on the distance between the imaging device conveying the illumination and the tissue. For example, the fluorescence excitation illumination source can be modulated for dynamically varying pulse width between 10 microseconds-8.342 milliseconds at 120 Hz based on distance. As the illumination source gets farther away from the tissue, the illumination period is dynamically set to be longer to increase exposure. In use cases in which the imaging device working distance can vary significantly, this imaging system may provide some technical advantages comparing to the imaging system utilizing the example modulation scheme in FIGS. 6A-B, which may provide fixed illumination periods of the fluorescence excitation illumination source and thus have weaker performance as the distance increases. In such use cases, the imaging system may also provide some technical advantages comparing to the imaging system with the always-on illumination scheme because, when the illumination source is close to the tissue, the illumination period is automatically set to be shorter so as to not create any false perception of fluorescence signals. Accordingly, the imaging system utilizing the modulation scheme in FIGS. 8A-B can keep the fluorescence signals consistent with any distance of operation.

Further, when the white-light illumination source is on, the fluorescence excitation illumination source is also on and the illumination period of the fluorescence excitation illumination source (e.g., the second illumination period) extends beyond the illumination period of the white-light illumination source (e.g., the first illumination period). As described herein, the extended period overlaps with the exposure of the next fluorescence frame such that the exposure time for the fluorescence frame is increased. This system may provide some technical advantages, for example, for imaging a relatively weak fluorescence signal compared to the imaging system utilizing the example modulation scheme in FIGS. 6A-B, in which the fluorescence excitation illumination source is not on when the white-light illumination source is on.

Accordingly, examples of the present disclosure can improve stability and consistency of the fluorescence images. In some examples, a user can select among a plurality of illumination schemes including the always-on illumination scheme and the various examples of modulation schemes described herein. In some examples, one particular exemplary modulation scheme can be set as a default for the imaging system.

Accordingly, described herein are exemplary devices, apparatuses, systems, methods, and non-transitory storage media for medical imaging. More generally are described exemplary devices, systems, and methods for modulating a fluorescence excitation illumination source. The systems, devices, and methods may be used for imaging tissue of a subject, such as in endoscopic imaging procedures and in open-field surgical procedures. An endoscopic imager may be pre-inserted into a subject prior to start of the imaging procedure. Imaging may be performed pre-operatively, intra-operatively, post-operatively, and during diagnostic imaging sessions and procedures. While some of the techniques are described with respect to a certain type of imager (e.g., a rolling shutter imager, a global shutter imager), it should be appreciated that the techniques can be applied in any type of imager. Further, the techniques can be applied in non-surgical or non-medical uses.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

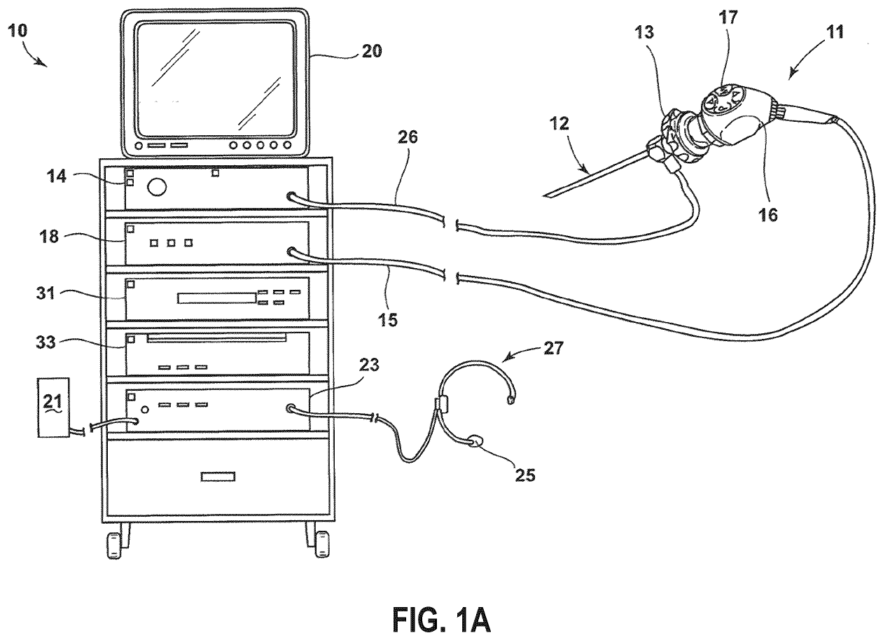

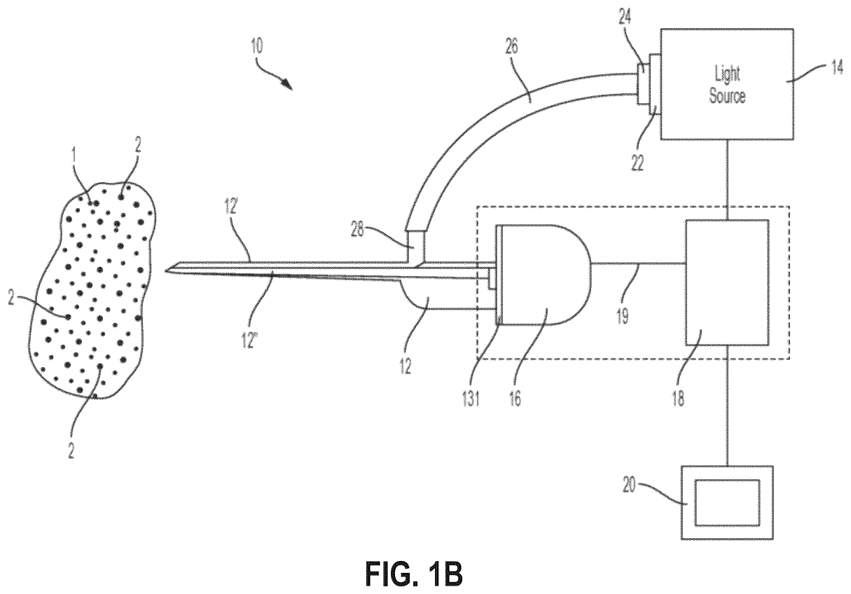

FIG. 1A shows an example of an endoscopic imaging system 10, which includes a scope assembly 11 which may be utilized in endoscopic procedures. The scope assembly 11 incorporates an endoscope or scope 12 which is coupled to a camera head 16 by a coupler 13 located at the distal end of the camera head 16. Light is provided to the scope by a light source 14 via a light guide 26, such as a fiber optic cable. The camera head 16 is coupled to a camera control unit (CCU) 18 by an electrical cable 15. The CCU 18 is connected to, and communicates with, the light source 14. Operation of the camera 16 is controlled, in part, by the CCU 18. The cable 15 conveys video image and/or still image data from the camera head 16 to the CCU 18 and may convey various control signals bi-directionally between the camera head 16 and the CCU 18.

A control or switch arrangement 17 may be provided on the camera head 16 for allowing a user to manually control various functions of the system 10, which may include switch from one imaging mode to another, as discussed further below. Voice commands may be input into a microphone 25 mounted on a headset 27 worn by the practitioner and coupled to the voice-control unit 23. A hand-held control device 29, such as a tablet with a touch screen user interface or a PDA, may be coupled to the voice control unit 23 as a further control interface. In the illustrated example, a recorder 31 and a printer 33 are also coupled to the CCU 18. Additional devices, such as an image capture and archiving device, may be included in the system 10 and coupled to the CCU 18. Video image data acquired by the camera head 16 and processed by the CCU 18 is converted to images, which can be displayed on a monitor 20, recorded by recorder 31, and/or used to generate static images, hard copies of which can be produced by the printer 33.

FIG. 1B shows an example of a portion of the endoscopic system 10 being used to illuminate and receive light from an object 1, such as a surgical site of a patient. The object 1 may include fluorescent markers 2, for example, as a result of the patient being administered a fluorescence imaging agent. The fluorescent imaging agent may have been pre-administered prior to start of the imaging procedure. The fluorescent markers 2 may be comprised of, for example, indocyanine green (ICG).

The light source 14 can generate visible illumination light (such as any combination of red, green, and blue light) for generating visible (e.g., white light) images of the target object 1 and can also produce fluorescence excitation illumination light for exciting the fluorescent markers 2 in the target object for generating fluorescence images. Illumination light is transmitted to and through an optic lens system 22 which focuses light onto a light pipe 24. The light pipe 24 may create a homogeneous light, which is then transmitted to the fiber optic light guide 26. The light guide 26 may include multiple optic fibers and is connected to a light post 28, which is part of the endoscope 12. The endoscope 12 includes an illumination pathway 12′ and an optical channel pathway 12″.

The endoscope 12 may include a notch filter 131 that allows some or all (preferably, at least 80%) of fluorescence emission light (e.g., in a wavelength range of 830 nm to 870 nm) emitted by fluorescence markers 2 in the target object 1 to pass therethrough and that allows some or all (preferably, at least 80%) of visible light (e.g., in the wavelength range of 400 nm to 700 nm), such as visible illumination light reflected by the target object 1, to pass therethrough, but that blocks substantially all of the fluorescence excitation light (e.g., infrared light having a wavelength of 808 nm) that is used to excite fluorescence emission from the fluorescent marker 2 in the target object 1. The notch filter 131 may have an optical density of OD5 or higher. In some examples, the notch filter 131 can be located in the coupler 13.

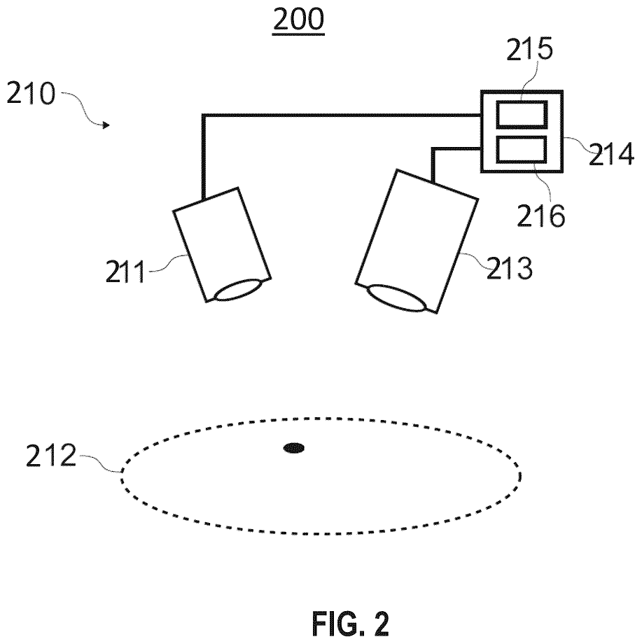

FIG. 2 illustrates an exemplary open field imaging system in accordance with some examples. FIG. 2 illustrates a schematic view of an illumination and imaging system 210 that can be used in open field surgical procedures. As may be seen therein, the system 210 may include an illumination module 211, an imaging module 213, and a video processor/illuminator (VPI) 214. The VPI 214 may include an illumination source 215 to provide illumination to the illumination module 211 and a processor assembly 216 to send control signals and to receive data about light detected by the imaging module 213 from a target 212 illuminated by light output by the illumination module 211. In one variation, the video processor/illuminator 214 may comprise a separately housed illumination source 215 and the processor assembly 216. In one variation, the video processor/illuminator 214 may comprise the processor assembly 216 while one or more illumination sources 215 are separately contained within the housing of the illumination module 211. The illumination source 215 may output light at different waveband regions, e.g., white (RGB) light, excitation light to induce fluorescence in the target 212, a combination thereof, and so forth, depending on characteristics to be examined and the material of the target 212. Light at different wavebands may be output by the illumination source 215 simultaneously, sequentially, or both. The illumination and imaging system 210 may be used, for example, to facilitate medical (e.g., surgical) decision making e.g., during a surgical procedure. The target 212 may be a topographically complex target, e.g., a biological material including tissue, an anatomical structure, other objects with contours and shapes resulting in shadowing when illuminated, and so forth. The VPI 214 may record, process, display, and so forth, the resulting images and associated information.

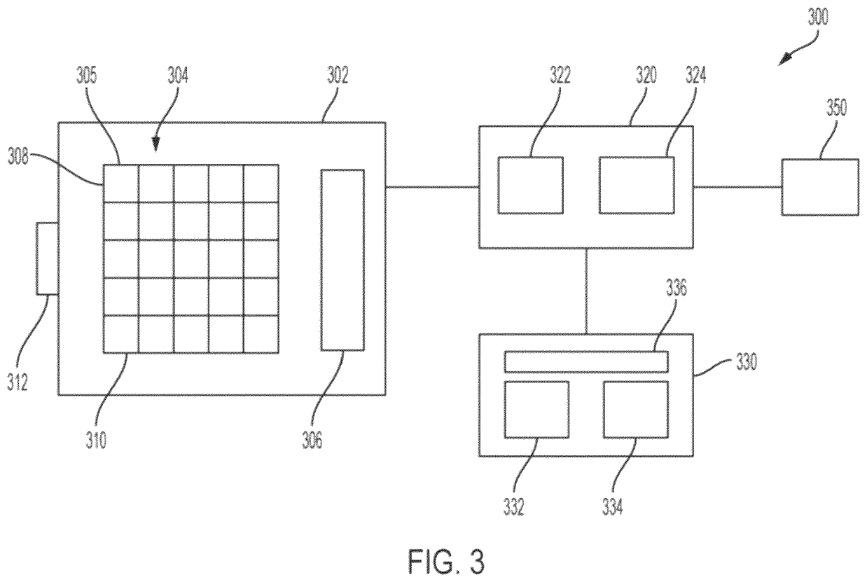

FIG. 3 schematically illustrates an exemplary imaging system 300 that employs an electronic imager 302 to generate images (e.g., still and/or video) of a target object, such as a target tissue of a patient, according to some examples. The imager 302 may be a rolling shutter imager (e.g., CMOS sensors) or a global shutter imager (e.g., CCD sensors). System 300 may be used, for example, for the endoscopic imaging system 10 of FIG. 1A. The imager 302 includes a CMOS sensor 304 having an array of pixels 305 arranged in rows of pixels 308 and columns of pixels 310. The imager 302 may include control components 306 that control the signals generated by the CMOS sensor 304. Examples of control components include gain circuitry for generating a multi-bit signal indicative of light incident on each pixel of the sensor 304, one or more analog-to-digital converters, one or more line drivers to act as a buffer and provide driving power for the sensor 304, row circuitry, and timing circuitry. A timing circuit may include components such as a bias circuit, a clock/timing generation circuit, and/or an oscillator. Row circuitry may enable one or more processing and/or operational tasks such as addressing rows of pixels 308, addressing columns of pixels 310, resetting charge on rows of pixels 308, enabling exposure of pixels 305, decoding signals, amplifying signals, analog-to-digital signal conversion, applying timing, read out and reset signals and other suitable processes or tasks. Imager 302 may also include a mechanical shutter 312 that may be used, for example, to control exposure of the image sensor 304 and/or to control an amount of light received at the image sensor 304.

One or more control components may be integrated into the same integrated circuit in which the sensor 304 is integrated or may be discrete components. The imager 302 may be incorporated into an imaging head, such as camera head 16 of system 10.

One or more control components 306, such as row circuitry and a timing circuit, may be electrically connected to an imaging controller 320, such as camera control unit 18 of system 10. The imaging controller 320 may include one or more processors 322 and memory 324. The imaging controller 320 receives imager row readouts and may control readout timings and other imager operations, including mechanical shutter operation. The imaging controller 320 may generate image frames, such as video frames from the row and/or column readouts from the imager 302. Generated frames may be provided to a display 350 for display to a user, such as a surgeon.

The system 300 in this example includes a light source 330 for illuminating a target scene. The light source 330 is controlled by the imaging controller 320. The imaging controller 320 may determine the type of illumination provided by the light source 330 (e.g., white light, fluorescence excitation light, or both), the intensity of the illumination provided by the light source 330, and or the on/off times of illumination in synchronization with rolling shutter operation. The light source 330 may include a first light generator 332 for generating light in a first wavelength and a second light generator 334 for generating light in a second wavelength. For example, in some examples, the first light generator 332 is a white light generator, which may be comprised of multiple discrete light generation components (e.g., multiple LEDs of different colors), and the second light generator 334 is a fluorescence excitation light generator, such as a laser diode.

The light source 330 includes a controller 336 for controlling light output of the light generators. The controller 336 may be configured to provide pulse width modulation of the light generators for modulating intensity of light provided by the light source 330, which can be used to manage over-exposure and under-exposure. In some examples, nominal current and/or voltage of each light generator remains constant and the light intensity is modulated by switching the light generators (e.g., LEDs) on and off according to a pulse width control signal. In some examples, a PWM control signal is provided by the imaging controller 336. This control signal can be a waveform that corresponds to the desired pulse width modulated operation of light generators.

The imaging controller 320 may be configured to determine the illumination intensity required of the light source 330 and may generate a PWM signal that is communicated to the light source 330. In some examples, depending on the amount of light received at the sensor 304 and the integration times, the light source may be pulsed at different rates to alter the intensity of illumination light at the target scene. The imaging controller 320 may determine a required illumination light intensity for a subsequent frame based on an amount of light received at the sensor 304 in a current frame and/or one or more previous frames. In some examples, the imaging controller 320 is capable of controlling pixel intensities via PWM of the light source 330 (to increase/decrease the amount of light at the pixels), via operation of the mechanical shutter 312 (to increase/decrease the amount of light at the pixels), and/or via changes in gain (to increase/decrease sensitivity of the pixels to received light). In some examples, the imaging controller 320 primarily uses PWM of the illumination source for controlling pixel intensities while holding the shutter open (or at least not operating the shutter) and maintaining gain levels. The controller 320 may operate the shutter 312 and/or modify the gain in the event that the light intensity is at a maximum or minimum and further adjustment is needed.

As described herein, the illumination source may or may not be separate from the imaging device in an imaging system. In some endoscopic imaging systems, the illumination source may be separate from the imaging device; while in some endoscopic imaging systems, the illumination source may be housed within the imaging device (e.g., one or more LEDs may be housed within the camera head). In some open-field imaging systems, the camera head may route illumination itself. In some examples, an imaging device can be an endoscope combined with a connected camera head (such as connected by a coupler or directly to the endoscope). In some examples, an imaging device can be a single camera head such as an open field imaging device.

### Deficiencies of the Always-on Fluorescence Excitation Illumination Scheme

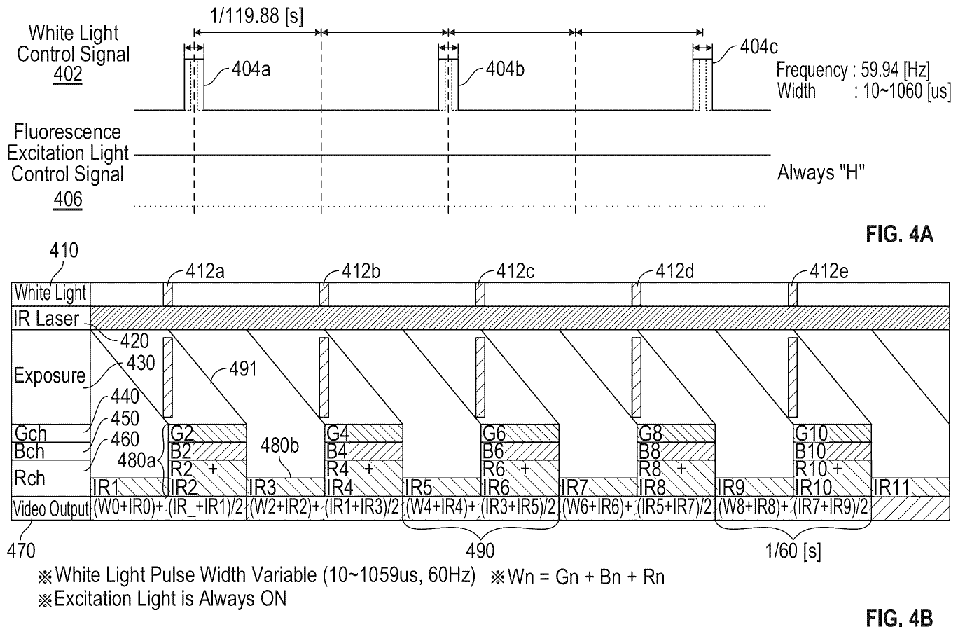

FIGS. 4A and 4B illustrate operations of an imaging system in which the fluorescence excitation illumination source is set to be always on without the option to modulate the source. FIG. 4A illustrates a control signal 402 for controlling a white-light illumination source of the imaging system and a control signal 406 for controlling a fluorescence excitation illumination source of the imaging system. In FIG. 4A, a pulse in the control signal indicates that the illumination source is on to illuminate a tissue of a subject, and the width of the pulse indicates the length of the illumination period.

With reference to FIG. 4A, the white-light illumination source is on during illumination periods 404a, 404b, 404c, etc., while being off otherwise. Further, the fluorescence excitation illumination source is always on during the imaging session. In the depicted example, the white-light illumination source is configured to provide illumination periods at 59.94 Hz (i.e., about 60 Hz), and the length of each illumination period is configured to be between 10 microseconds to 1060 microseconds (i.e., about 1 millisecond).

FIG. 4B illustrates a timing diagram of an imaging system, in accordance with some examples. The timing of the operation of the white-light illumination source is shown in row 410, and the timing of the operation of the fluorescence excitation illumination source is shown in row 420. In the depicted example, the white-light illumination source is configured to be on during illumination periods 412a, 412b, 412c, 412d, 412e, etc., at a frequency (i.e., 60 Hz) equaling half the frame rate of the rolling shutter imager (i.e., 120 Hz). Further, the fluorescence excitation illumination source is configured to be always on, as indicated by the row 420.

Further with reference to FIG. 4B, row 440 indicates the imaging data acquired in the green channel; row 450 indicates the imaging data acquired in the blue channel; row 460 indicates the imaging data acquired in the red channel. As shown, the system illuminates the tissue of the subject with the white-light illumination source for an illumination period 412a to accumulate charge at a plurality of rows of pixels of the rolling shutter imager. After the first illumination period 412a, the system sequentially reads the accumulated charge at the plurality of rows of pixels from a first row to a last row of the plurality of rows to produce a set of imaging data 480a, which comprises imaging data G2 in the green channel, imaging data B2 in the blue channel, and imaging data R2 and IR2 (i.e., fluorescence imaging data due to the always-on fluorescence excitation illumination source) in the red channel. The data 480a can be used to generate a white-light frame having a fluorescence component IR2.

As discussed below, while FIG. 4B illustrates operations of an imaging system conveying infrared illumination to obtain IR fluorescence imaging data, it should be appreciated that the imaging system is merely exemplary and that other imaging systems with other fluorophore configurations (e.g., blue light imaging, other fluorophores that emit in other wavelength ranges) may operate according to the illumination scheme in FIG. 4B. In these other imaging systems, the fluorescence image data may be acquired in a different channel than the red channel, or even on multiple channels.

Further, in the depicted example in FIG. 4B (and similarly in FIGS. 6B and 8B), the timing for each illumination period is shown as to the left of (i.e., prior to) the starting time of the corresponding white-light RGB channel readout period, rather than starting at the same time as the start of the readout period. For example, the timing for the illumination period 412a is shown to the left of the starting time of the corresponding white-light RGB channel readout period (as indicated by the diagonal line 491), rather than starting at the same time as the start of the period. This is due to the fact that, in this particular example, each illumination period is configured to spread into the previous frame over the last few lines to increase exposure time for the image frames of the corresponding readout period (e.g., to avoid reaching full illumination strength later than the start of the readout period), and the top and bottom of each image can be masked to remove the resulting artifacts. It should be appreciated that this timing configuration is merely exemplary, and that, in some examples, the timing for each illumination period may be aligned with the start of the corresponding readout period.

Further with reference to FIG. 4B, between the illumination period 412a and the illumination period 412b, the white-light illumination source is off but the fluorescence excitation illumination source is always on. As such, the system can sequentially read the accumulated charge to produce imaging data 480b, which comprises only fluorescence image data IR3 acquired in the red channel. The imaging data 480b (also referred to as IR3 in FIG. 4B) can be used to generate a fluorescence frame.

Further sets of imaging data W4 (comprising G4, B4, R4), IR4, IR5, W6 (comprising G6, B6, R6), IR6, IR7, W8 (comprising G8, B8, R8), IR8, IR9, W10 (comprising G10, B10, R10), IR10, IR11, etc., are generated in a similar manner as described above. The system can generate image frames based on these sets of imaging data. In the depicted example, the imaging system produces about 60 fluorescence image frames and 60 white-light image frames per second, with each frame exposed to either only the fluorescence excitation illumination source or both illumination sources at the same time. In other words, the rolling shutter imager (e.g., in the camera head) outputs image frames at the sensor frame rate of 120 Hz, which are then processed in the camera control unit to generate the display output. In the depicted example, a blended image is generated every 1/60s (i.e., at 60 Hz) as shown in row 470 (video output). Thus, if the camera control unit is configured to output the blended images, the display output frame rate would be 60 fps. Each blended frame can be based on a combination of one or more white-light images and one or more fluorescence images. For example, the blended frame 490 is a combination of previously acquired W4 and IR4 and an average of two previously acquired fluorescence image frames IR3 and IR5.

The imaging system in FIGS. 4A-B have a number of deficiencies. In the imaging system, the fluorescence excitation illumination source is constantly on, thus providing maximum exposure time for the fluorescence image frames and providing maximum fluorescence excitation strength over the entire range of working distances (e.g., the distance from the front lens element of the imaging objective (e.g., the front lens element of the endoscope) to the specimen/tissue being observed). However, when the tissue to be imaged is close to the imaging device that is emitting the illumination (e.g., at the minimum working distance), the fluorescence excitation strength can be excessively high and cause an intense fluorescence emission that in turn generates a strong fluorescence signal in the camera of the imaging device. Because each white-light image frame and/or blended image generated by the imaging system has a fluorescence component, the strong fluorescence signal generated by the emission may reduce the background/white-light average luminance and contaminate the image frame. Specifically, because there is some cross-contamination of the imaging signal from each color channel into other color channels, if the fluorescence signal is abnormally high, it may contaminate other color channels. For example, the entire scene in the image frame may appear green if the fluorescence signal is represented in green and create a perception of false fluorescence signals in the camera of the imaging device. This is problematic for both open-field cameras and endoscopic cameras. For example, because endoscopes can get very close to the tissue and can be moved in and out of an area quickly during surgeries, the distance between the endoscope and the imaged tissue can vary quickly, thus resulting in false fluorescence signals.

### Examples of Modulated Fluorescence Excitation Illumination Source

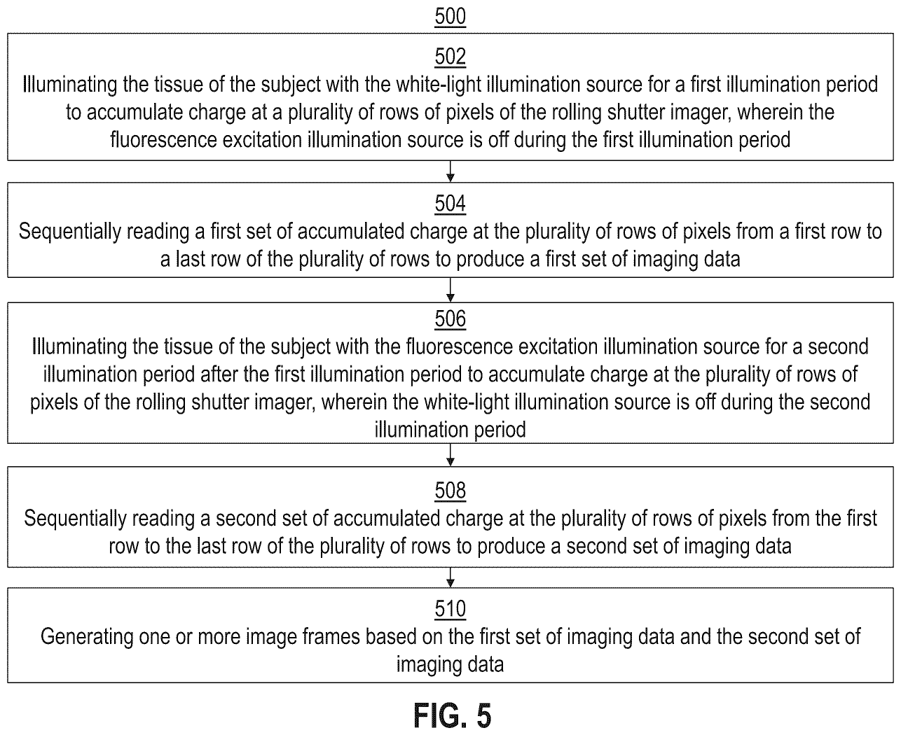

FIG. 5 provides an exemplary method 500 for imaging tissue of a subject, in accordance with some examples. Method 500 may be performed by an imaging system that comprises a rolling shutter imager, a fluorescence excitation illumination source, and a white-light illumination source. The fluorescence excitation illumination source can comprise an infrared light, a blue-light illumination source, or any combination thereof. The white-light illumination source can comprise one or more LEDs. The rolling shutter imager can be part of an endoscopic imager or an open-field imager and may comprise a CMOS sensor. The imaging system can be the imaging system 300 of FIG. 3, which has a rolling shutter imager (e.g., rolling shutter imager 302 of system 300) and a light source (e.g., light source 330 of system 300) that can comprise a fluorescence excitation illumination source and/or a white-light illumination source.

In process 500, some blocks are optionally combined, the order of some blocks is optionally changed, and some blocks are optionally omitted. In some examples, additional steps may be performed in combination with the process 500. Accordingly, the operations as illustrated (and described in greater detail below) are exemplary by nature and, as such, should not be viewed as limiting.

At block 502, an exemplary system illuminates the tissue of the subject with the white-light illumination source for a first illumination period to accumulate charge at a plurality of rows of pixels of the rolling shutter imager. The fluorescence excitation illumination source is off during the first illumination period. At block 504, the system sequentially reads a first set of accumulated charge at the plurality of rows of pixels from a first row to a last row of the plurality of rows to produce a first set of imaging data. At block 506, the system illuminates the tissue of the subject with the fluorescence excitation illumination source for a second illumination period after the first illumination period to accumulate charge at the plurality of rows of pixels of the rolling shutter imager. The white-light illumination source is off during the second illumination period. At block 508, the system sequentially reads a second set of accumulated charge at the plurality of rows of pixels from the first row to the last row of the plurality of rows to produce a second set of imaging data. At block 510, the system generates one or more image frames based on the first set of imaging data and the second set of imaging data.

In some examples, the fluorescence excitation illumination source and the white-light illumination source are configured to alternately provide illumination periods to the tissue of the subject. Exemplary operations of the imaging system are shown in FIGS. 6A-B, which is described in detail below.

In some examples, the length of each illumination period of the white-light illumination source is a variable configured to not exceed a first maximum value (e.g., about 1 millisecond). The variable may be dynamically determined based on the distance between the imaging device conveying the illumination and the subject/scene being visualized. The distance can be measured based on the average luminance/intensity of the previous acquired frame. This way, if the illumination source is closer, the illumination period would be shorter because not much light is needed. In contrast, if the illumination source is far, the illumination period is longer to increase the exposure. It should be appreciated by one of ordinary skill in the art that the first maximum value may vary depending on the speed of the sensor used in the system.

In some examples, the length of each illumination period of the fluorescence excitation illumination source is a fixed value; alternatively (not depicted in FIGS. 6A-B), the length of each illumination period of the fluorescence excitation illumination source is a variable configured to not exceed a second maximum value.

In some examples, the fluorescence excitation illumination source is configured to provide illumination periods at a first frequency. The first frequency may correspond to the frame rate of the rolling shutter imager. For example, the first frequency may be half the frame rate of the rolling shutter imager. Further, the white-light illumination source is configured to provide illumination periods at a second frequency. The second frequency may correspond to the frame rate of the rolling shutter imager. For example, the second frequency may be half the frame rate of the rolling shutter imager.

In some examples, the one or more image frames generated by the process 500 comprise a white-light image frame based on the first set of imaging data, a fluorescence image frame based on the second set of imaging data, or both. The one or more image frames can further comprise a blended image frame based on the fluorescence image frame and the white-light image frame. The fluorescence image frame may be overlaid on the white-light image frame in the blended image frame. The blended image frame may be derived from colorizing the white-light image frame based on the fluorescence image frame. In some examples, the system adds the one or more image frames to a video stream.

FIG. 6A illustrates exemplary operations of an exemplary imaging system performing the process 500, in accordance with some examples. FIG. 6A illustrates a control signal 602 for controlling a white-light illumination source of the imaging system and a control signal 606 for controlling a fluorescence excitation illumination source of the imaging system. In FIG. 6A, a pulse in the control signal indicates that the illumination source is on to illuminate a tissue of a subject, and the width of the pulse indicates the length of the illumination period. It should be appreciated that FIG. 6A illustrates the frequencies of the white-light illumination periods and the fluorescence excitation illumination periods and the general correspondence between the two, but does not illustrate the precise starting times and ending times of the illumination periods.

With reference to FIG. 6A, the exemplary system first illuminates the tissue of the subject with the white-light illumination source for a first illumination period 604a to accumulate charge at a plurality of rows of pixels of the rolling shutter imager. As indicated by the control signal 606, during the first illumination period 604a, the fluorescence excitation illumination source is off. After the first illumination period 604a, the system sequentially reads a first set of accumulated charge at the plurality of rows of pixels from a first row to a last row of the plurality of rows to produce a first set of imaging data.

With reference to FIG. 6A, the system then illuminates the tissue of the subject with the fluorescence excitation illumination source for a second illumination period 608a after the first illumination period 604a to accumulate charge at the plurality of rows of pixels of the rolling shutter imager. As indicated by the control signal 602, the white-light illumination source is off during the second illumination period 608a. After the second illumination period 608a, the system sequentially reads a second set of accumulated charge at the plurality of rows of pixels from the first row to the last row of the plurality of rows to produce a second set of imaging data. The system generates one or more image frames based on the first set of imaging data and the second set of imaging data. The generation of the one or more image frames is described in detail herein with reference to FIG. 6B.

In the depicted example in FIG. 6A, the fluorescence excitation illumination source and the white-light illumination source are configured to alternately provide illumination periods to the tissue of the subject. As shown, the white-light illumination source is on during illumination periods 604a, 604b, 604c, etc., while being off otherwise. Similarly, the fluorescence excitation illumination source is on during illumination periods 608a, 608b, etc., while being off otherwise. The illumination periods of the white-light illumination source and the illumination periods of the fluorescence excitation illumination source are offset such that the tissue is first illuminated only by the white-light illumination source in 604a, then illuminated only by the fluorescence excitation illumination source in 608a, then illuminated only by the white-light illumination source in 604b, then illuminated only by the fluorescence excitation illumination source in 608b, then illuminated only by the white-light illumination source in 604c, and so on.

As indicated by the control signal 602, each illumination period of the white-light illumination source may be a variable configured to not exceed a first maximum value. The variable may be dynamically determined based on the distance between the imaging device conveying the illumination and the subject/scene being visualized. The distance can be measured based on the average luminance/intensity of the previous acquired frame. This way, if the illumination source is closer, the illumination period would be shorter because not much light is needed. In contrast, if the illumination source is far, the illumination period is longer to increase the exposure. In the depicted example, the length of the illumination periods of the white-light illumination source can be between 10 microseconds to 1060 microseconds (i.e., about 1 millisecond). In other words, the first maximum value may be about 1 millisecond.

As indicated by the control signal 606, each illumination period of the fluorescence excitation illumination source may be a fixed value. In the depicted example, the length can be set to 1060 microseconds (i.e., about 1 millisecond). Alternatively, the length can be configured to be a variable configured to not exceed a second maximum value.

The fluorescence excitation illumination source can be configured to provide illumination periods at a first frequency. The first frequency may correspond to the frame rate of the rolling shutter imager. For example, the first frequency may be half the frame rate of the rolling shutter imager (e.g., the imaging sensor frame rate output from the camera head). In the depicted example, the first frequency is 59.94 Hz (i.e., about 60 Hz), which is half the frame rate of the rolling shutter imager (i.e., about 120 Hz). Further, the white-light illumination source is configured to provide illumination periods at a second frequency. The second frequency may correspond to the frame rate of the rolling shutter imager. For example, the second frequency may be half the frame rate of the rolling shutter imager. In the depicted example, the second frequency is 59.94 Hz (i.e., about 60 Hz), the same as the fluorescence excitation illumination source.

FIG. 6B illustrates a timing diagram, or modulation scheme, of an exemplary imaging system, in accordance with some examples. The timing of the operation of the white-light illumination source is shown in row 610, and the timing of the operation of the fluorescence excitation illumination source is shown in row 620. In the depicted example, the white-light illumination source is configured to be on during illumination periods 612a, 612b, 612c, 612d, 612e, etc., at a frequency (i.e., 60 Hz) equaling half the frame rate of the rolling shutter imager (i.e., 120 Hz). Further, the fluorescence excitation illumination source is configured to be on during illumination periods 622a, 622b, 622c, 622d, 622e, etc., at a frequency (i.e., 60 Hz) equaling half the frame rate of the rolling shutter imager (i.e., 120 Hz).

The illumination periods of the white-light illumination source and the illumination periods of the fluorescence excitation illumination source are offset by 1/120s such that, together, they alternately illuminate a tissue of a subject at a frequency of 120 Hz. Accordingly, as shown by row 630, the exposure window occurs at a frequency of 120 Hz.

Further with reference to FIG. 6B, row 640 indicates the imaging data acquired in the green channel; row 650 indicates the imaging data acquired in the blue channel; row 660 indicates the imaging data acquired in the red channel. As shown, the system illuminates the tissue of the subject with the white-light illumination source for an illumination period 612a to accumulate charge at a plurality of rows of pixels of the rolling shutter imager. After the first illumination period 612a, the system sequentially reads the accumulated charge at the plurality of rows of pixels from a first row to a last row of the plurality of rows to produce a set of imaging data 680a, which comprises imaging data G2 in the green channel, imaging data B2 in the blue channel, and imaging data R2 in the red channel. The imaging data 680 (also referred to as W2 in FIG. 6B) can be used to generate a white-light frame.

Further with reference to FIG. 6B, the system then illuminates the tissue of the subject with the fluorescence excitation illumination source for an illumination period 622a to accumulate charge at the plurality of rows of pixels of the rolling shutter imager. After the second illumination period 622a, the system sequentially reads the accumulated charge at the plurality of rows of pixels from the first row to the last row of the plurality of rows to produce imaging data 680b, which comprises only fluorescence image data IR3 acquired in the red channel. The imaging data 680b (also referred to as IR3 in FIG. 6B) can be used to generate a fluorescence frame. While FIG. 6B illustrates operations of an imaging system conveying infrared illumination to obtain IR fluorescence imaging data, it should be appreciated that the imaging system is merely exemplary and that other imaging systems with other fluorophore configurations (e.g., blue light imaging, other fluorophores that emit in other wavelength ranges) may operate according to the illumination scheme in FIG. 6B. In these other imaging systems, the fluorescence image data may be acquired in a different channel than the red channel, or even on multiple channels.

Further sets of imaging data W4 (comprising G4, B4, R4), IR5, W6 (comprising G6, B6, R6), IR7, W8 (comprising G8, B8, R8), IR9, W10 (comprising G10, B10, R10), IR11, etc., are generated in a similar manner as described above. The system can generate image frames based on these sets of imaging data. In the depicted example, the rolling shutter imager (e.g., in the camera head) outputs the sets of imaging data at the sensor frame rate of 120 Hz, which is then processed in the camera control unit to generate the display output. In the depicted example, as shown in row 670 (video output), a blended image is generated every 1/60s (i.e., at 60 Hz). Thus, if the camera control unit is configured to output the blended images, the display output frame rate would be 60 fps.

Each blended frame can be based on a combination of one or more white-light images and one or more fluorescence images. The blended image frame may be derived from colorizing a white-light image frame based on a fluorescence image frame. In the depicted example, a blended frame is a combination of a previously acquired white-light image and an average of two previously acquired fluorescence image frames. For example, the blended frame 690 is a combination of a previously acquired white-light image W4 and an average of two previously acquired fluorescence image frames IR3 and IR5. The average of the two fluorescence image frames may be overlaid on the white-light image. In some examples, the system adds the blended image frames to a video stream, which may be provided during a surgical procedure.

In some examples, the system can be configured to receive a user input indicative of which type of image data to display (e.g., white-light image data, fluorescence image data, blended image data) and update the display based on the user input. Accordingly, the system can allow the user to switch among multiple viewing modes, for example, intraoperatively.

The imaging system in FIGS. 6A-B may provide some technical advantages over the imaging system in FIGS. 4A-B. The fluorescence excitation illumination source is modulated to be on for about 1 millisecond at about 60 Hz 180° phase offset to white-light illumination source. By isolating the white-light illumination and the fluorescence excitation illumination, the imaging system can reduce cross-contamination and gain better isolation between frames and the resulting blended image can be clearer. Comparing to the imaging system in FIGS. 4-B (i.e., the always-on scheme), the imaging system in FIGS. 6A-B may provide better control and consistency of the fluorescence images. The imaging system can provide improved excitation illumination strength at the minimum working distance.

However, the imaging system in FIGS. 6A-B may have weaker performance as the surgical imaging device that is emitting the illumination gets farther away from the tissue. Because the fluorescence excitation illumination periods are set to a fixed value (e.g., only about one millisecond), if the imaging device gets far enough from the tissue, the imaging system may lose the fluorescence strength due to the distance and the fixed exposure time.

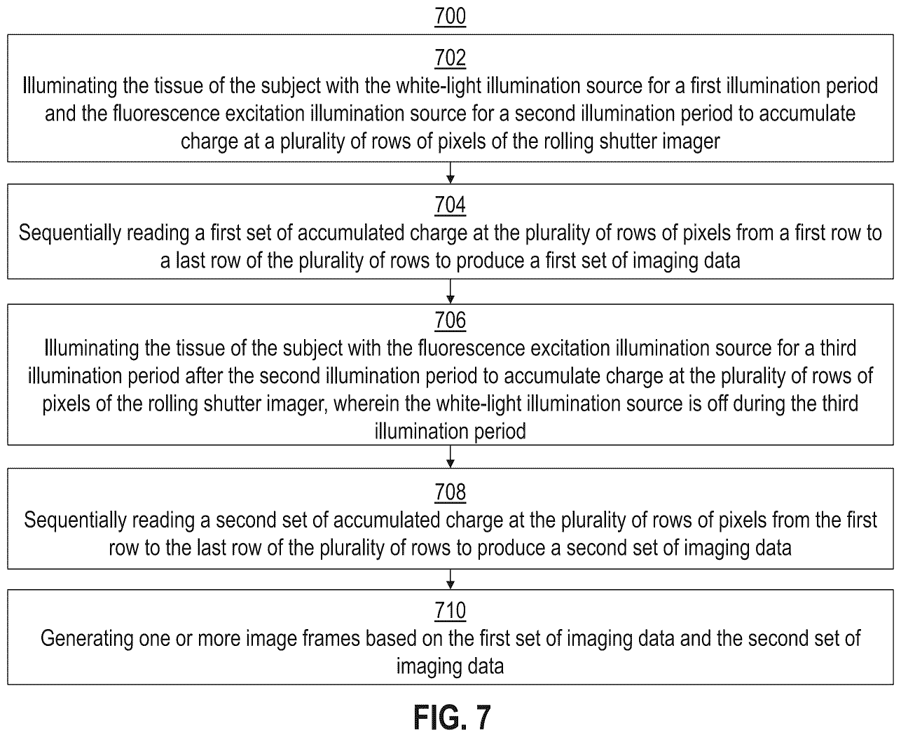

FIG. 7 provides an exemplary method 700 for imaging tissue of a subject, in accordance with some examples. Method 700 may be performed by an imaging system that comprises a rolling shutter imager, a fluorescence excitation illumination source, and a white-light illumination source. The fluorescence excitation illumination source can comprise an infrared light, a blue-light illumination source, or any combination thereof. The white-light illumination source can comprise one or more LEDs. The rolling shutter imager can be part of an endoscopic imager or an open-field imager and may comprise a CMOS sensor. The imaging system can be the imaging system 300 of FIG. 3, which has a rolling shutter imager (e.g., rolling shutter imager 302 of system 300) and a light source (e.g., light source 330 of system 300) that can comprise a fluorescence excitation illumination source and/or a white-light illumination source.

In process 700, some blocks are optionally combined, the order of some blocks is optionally changed, and some blocks are optionally omitted. In some examples, additional steps may be performed in combination with the process 700. Accordingly, the operations as illustrated (and described in greater detail below) are exemplary by nature and, as such, should not be viewed as limiting.

At block 702, an exemplary system illustrates the tissue of the subject with the white-light illumination source for a first illumination period and the fluorescence excitation illumination source for a second illumination period to accumulate charge at a plurality of rows of pixels of the rolling shutter imager. The first illumination period and the second illumination period may start at the same time, and the second illumination period may be longer than the first illumination period. At block 704, the system sequentially reads a first set of accumulated charge at the plurality of rows of pixels from a first row to a last row of the plurality of rows to produce a first set of imaging data. At block 706, the system illuminates the tissue of the subject with the fluorescence excitation illumination source for a third illumination period after the second illumination period to accumulate charge at the plurality of rows of pixels of the rolling shutter imager. The white-light illumination source is off during the third illumination period. At block 708, the system sequentially reads a second set of accumulated charge at the plurality of rows of pixels from the first row to the last row of the plurality of rows to produce a second set of imaging data. At block 710, the system generates one or more image frames based on the first set of imaging data and the second set of imaging data.

In some examples, the fluorescence excitation illumination source is configured to provide illumination periods to the tissue of the subject at a first frequency, and the white-light illumination source is configured to provide illumination during at least part of every other illumination period of the illumination periods of the fluorescence excitation illumination source. In some examples, the first frequency corresponds to the frame rate of the rolling shutter imager. For example, the first frequency may equal the frame rate of the rolling shutter imager.

In some examples, each illumination period of the white-light illumination source is a variable configured to not exceed a first maximum value (e.g., about 1 millisecond). In some examples, each illumination period of the fluorescence excitation illumination source is a variable based on the distance between the imaging device and the tissue. The distance may be determined based on a luminance value calculated based on the first set of imaging data.

In some examples, the one or more image frames generated by the process 700 comprise a white-light image frame based on the first set of imaging data, a fluorescence image frame based on the second set of imaging data, or both. The one or more image frames can further comprise a blended image frame based on the fluorescence image frame and the white-light image frame. The fluorescence image frame may be overlaid on the white-light image frame in the blended image frame. The blended image frame may be derived from colorizing the white-light image frame based on the fluorescence image frame. In some examples, the system adds the one or more image frames to a video stream.

FIG. 8A illustrates exemplary operations of an exemplary imaging system performing the process 700, in accordance with some examples. FIG. 8A illustrates a control signal 802 for controlling a white-light illumination source of the imaging system and a control signal 806 for controlling a fluorescence excitation illumination source of the imaging system. In FIG. 8A, a pulse indicates that the illumination source is on to illuminate a tissue of a subject, and the width of the pulse indicates the length of the illumination period. As discussed above, FIG. 8A illustrates the frequencies of the white-light illumination periods and the fluorescence excitation illumination periods and the general correspondence between the two, but does not depict the precise starting times and ending times of the illumination periods. For example, as described in more detail with reference to FIG. 8B, the first illumination period 804a and the second illumination period 808a may start at the same time, and the second illumination period 808a may be longer than the first illumination period 804a, which is not depicted in FIG. 8A.

With reference to FIG. 8A, the exemplary system first illuminates the tissue of the subject with the white-light illumination source for a first illumination period 804a and with the fluorescence excitation illumination source for a second illumination period 808a to accumulate charge at a plurality of rows of pixels of the rolling shutter imager. The system then sequentially reads the accumulated charge at the plurality of rows of pixels from a first row to a last row of the plurality of rows to produce a first set of imaging data. The system illuminates the tissue of the subject with the fluorescence excitation illumination source only for a third illumination period 808b after the second illumination period 808a to accumulate charge at the plurality of rows of pixels of the rolling shutter imager. The white-light illumination source is off during the third illumination period 808b, as shown by the control signal 802. The system then sequentially reads a second set of accumulated charge at the plurality of rows of pixels from the first row to the last row of the plurality of rows to produce a second set of imaging data. The system can then generate one or more image frames based on the first set of imaging data and the second set of imaging data.

Further with reference to FIG. 8A, the fluorescence excitation illumination source is configured to provide illumination periods to the tissue of the subject at a first frequency, and the white-light illumination source is configured to provide illumination during at least part of every other illumination period of the illumination periods of the fluorescence excitation illumination source. The first frequency corresponds to the frame rate of the rolling shutter imager. For example, the first frequency may equal the frame rate of the rolling shutter imager. In the depicted example, the frame rate of the rolling shutter imager is about 120 Hz. As shown by the control signal 806, the fluorescence excitation illumination source is configured to provide illumination periods to the tissue of the subject at about 120 Hz. In contrast, as shown by control signal 802, the white-light illumination source is configured to provide illumination during at least part of every other illumination period of the illumination periods of the fluorescence excitation illumination source, at about 60 Hz.

As indicated by the control signal 802, the length of each illumination period of the white-light illumination source may be a variable configured to not exceed a first maximum value. The variable may be dynamically determined based on the distance between the imaging device conveying the illumination and the subject/scene being visualized. The distance can be measured based on the average luminance/intensity of the previous acquired frame. This way, if the illumination source is closer, the illumination period would be shorter because not much light is needed. In contrast, if the illumination source is far, the illumination period is longer to increase the exposure. In the depicted example, the length of the illumination periods of the white-light illumination source can be between 10 microseconds to 1060 microseconds (i.e., about 1 millisecond). In other words, the first maximum value may be about 1 millisecond.

As indicated by the control signal 806, the length of each illumination period (i.e., the pulse width) of the fluorescence excitation illumination source may be a variable dynamically based on a distance between the imaging device conveying the illumination and the tissue. If the distance is longer, the length of the illumination period is automatically set to be longer. On the other hand, if the distance is shorter, the length of the illumination period is automatically set to be shorter. In the depicted example, the length of the illumination periods of the fluorescence excitation illumination source can be configured to be within a range of 10 microseconds to 8342 microseconds. The distance can be determined, for example, based on a luminance value calculated based on the first set of imaging data. For example, the distance may be determined based on the average luminance of the previous white light frame. This is because the average luminance would change based on the distance, increasing if the distance is shorter and decreasing if the distance is longer. It should be appreciated that the distance may be determined using other means, such as a proximity sensor.

FIG. 8B illustrates a timing diagram, or modulation scheme, of an exemplary imaging system, in accordance with some examples. The timing of the operation of the white-light illumination source is shown in row 810, and the timing of the operation of the fluorescence excitation illumination source is shown in row 820. In the depicted example, the white-light illumination source is configured to be on during illumination periods 812a, 812b, 812c, 812d, 812e, etc., at a frequency (i.e., 60 Hz) equaling half the frame rate of the rolling shutter imager (i.e., 120 Hz). Further, the fluorescence excitation illumination source is configured to be on during illumination periods 822a, 822b, 822c, 822d, 822e, 822f, etc., at a frequency equaling the frame rate of the rolling shutter imager (i.e., 120 Hz). In the depicted example, the illumination periods 812a and 822a start at the same time, and the illumination period 822a extends beyond the illumination period 812a.

With reference to FIG. 8B, the white-light illumination source and the fluorescence excitation illumination source are both on during illumination periods 812a (which overlaps with the first portion of 822a), 812b (which overlaps with the first portion of 822c), 812c (which overlaps with the first portion of 822e), etc. The fluorescence excitation illumination source is additionally on during the remaining portions of 822a, 822c, 833e, etc., as well as illumination periods 822b, 822d, 822f, etc. Accordingly, the tissue is first illuminated by both the white-light illumination source and the fluorescence excitation illumination source in 812a and the overlapping portion of 822a, then illuminated only by the fluorescence excitation illumination source for the remaining duration of period 822a, then illuminated only by the fluorescence excitation illumination source in 822b, then illuminated by both illumination sources in 812b and the overlapping portion of 822c, then illuminated only by the fluorescence excitation illumination source for the remaining duration of period 822c, then illuminated only by the fluorescence excitation illumination source in 822d, and so on.

Further with reference to FIG. 8B, row 840 indicates the imaging data acquired in the green channel; row 850 indicates the imaging data acquired in the blue channel; row 860 indicates the imaging data acquired in the red channel. As shown, the system illuminates the tissue of the subject with both illumination sources for an illumination period 812a to accumulate charge at a plurality of rows of pixels of the rolling shutter imager. After the first illumination period 812a, the system sequentially reads the accumulated charge at the plurality of rows of pixels from a first row to a last row of the plurality of rows to produce a set of imaging data 880a, which comprises imaging data G2 in the green channel, imaging data B2 in the blue channel, and imaging data R2 and IR2 in the red channel. Additionally, because both illumination sources are on during the illumination window 812a, the system obtains fluorescence imaging data IR2 in the red channel. The data G2, B2, and R2 (also collectively referred to as W2 in FIG. 8B), plus IR2, can be used to generate a white-light frame with a fluorescence component. While FIG. 8B illustrates operations of an imaging system conveying infrared illumination to obtain IR fluorescence imaging data, it should be appreciated that the imaging system is merely exemplary and that other imaging systems with other fluorophore configurations (e.g., blue light imaging, other fluorophores that emit in other wavelength ranges) may operate according to the illumination scheme in FIG. 8B. In these other imaging systems, the fluorescence image data may be acquired in a different channel than the red channel, or even on multiple channels.

Further with reference to FIG. 8B, the system then illuminates the tissue of the subject with the fluorescence excitation illumination source for an illumination period 822b to accumulate charge at the plurality of rows of pixels of the rolling shutter imager. After the second illumination period 822b, the system sequentially reads the accumulated charge at the plurality of rows of pixels from the first row to the last row of the plurality of rows to produce imaging data 880b. The imaging data 880b is a result of both at least partially the illumination period 822a and the illumination period 822b and comprises only fluorescence image data IR3 acquired in the red channel. The imaging data 880b (also referred to as IR3 in FIG. 8B) can be used to generate a fluorescence frame.

Further sets of imaging data W4 (comprising G4, B4, R4), IR4, IR5, W6 (comprising G6, B6, R6), IR6, IR7, W8 (comprising G8, B8, R8), IR8, IR9, W10 (comprising G10, B10, R10), IR10, IR11, etc., are generated in a similar manner as described above. The system can generate image frames based on these sets of imaging data. In the depicted example, the rolling shutter imager (e.g., in the camera head) outputs the sets of imaging data at the sensor frame rate of 120 Hz, which is then processed in the camera control unit to generate the display output. In the depicted example, as shown in row 870 (video output), a blended image is generated every 1/60s (i.e., at 60 Hz). Thus, if the camera control unit is configured to output the blended images, the display output frame rate would be 60 fps.

Each blended frame can be based on a combination of one or more white-light images and one or more fluorescence images. The blended image frame may be derived from colorizing a white-light image frame based on a fluorescence image frame. For example, the blended frame 890 is a combination of previously acquired W4 and IR4 and an average of IR3 and IR5. The fluorescence signal may be overlaid on the white-light image. In some examples, the system adds the blended image frames to a video stream, which may be displayed during a surgical procedure.

The imaging system operating according to a modulation scheme such as the one depicted in FIGS. 8A-B may provide a number of technical advantages. First, the length of each illumination period of the fluorescence excitation illumination source is dynamically adjusted based on the distance between the imaging device conveying the illumination and the tissue. In the depicted examples in FIGS. 8A-B, the fluorescence excitation illumination source is modulated for dynamically varying pulse width between 10 microseconds-8.342 milliseconds at 120 Hz based on distance. As the imaging device conveying the illumination gets farther away from the tissue, the illumination period is dynamically set to be longer to increase exposure. The imaging system may provide some technical advantages comparing to the imaging system in FIGS. 6A-B, which provides fixed illumination periods of the fluorescence excitation illumination source and thus has weaker performance as the distance increases. The imaging system may provide some technical advantages comparing to the imaging system in FIGS. 4A-B (i.e., the always-on scheme) because, when the imaging device conveying the illumination is close to the tissue, the illumination period is automatically set to be shorter so as to not create any false perception of fluorescent signals. Accordingly, the imaging system in FIGS. 8A-B may keep the fluorescence signals consistent with any distance of operation.

Further, in the imaging system operating according to the exemplary modulation scheme in FIGS. 8A-B, when the white-light illumination source is on, the fluorescence excitation illumination source is also on and the illumination period of the fluorescence excitation illumination source (e.g., 822a) extends beyond the illumination period of the white-light illumination source (e.g., 812a). As shown in FIG. 8B, the extended period overlaps onto the next fluorescence frame such that the exposure time for the fluorescence frame is increased. In other words, IR3 is the result of both at least a portion of the illumination period 822a and at least a portion of the illumination period 822b, as indicated by the circle 823. This system may provide some technical advantages comparing to the imaging system in FIGS. 6A-B, in which the fluorescence excitation illumination source is not on when the white-light illumination source is on.

Accordingly, the imaging system operating according to the exemplary modulation scheme in FIGS. 8A-B may improve stability and consistency of the fluorescence images. In some examples, a user can select among a plurality of illumination schemes including the always-on illumination scheme in FIGS. 4A-B, the examples of a modulation scheme in FIGS. 6A-B, and/or the examples of the modulation scheme in FIGS. 8A-B. In some examples, a modulation scheme such as depicted in the examples in FIGS. 8A-B may be set to be the default.

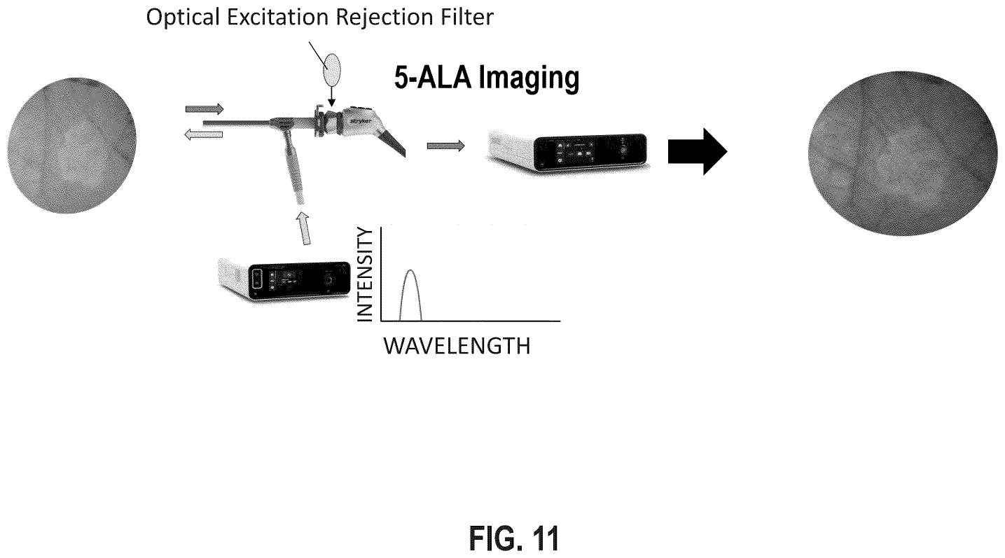

The examples of modulation schemes described herein can be used by imaging systems with other fluorophore configurations (e.g., blue light imaging, any other fluorophores that emit in other wavelength ranges). FIG. 11 illustrates an exemplary blue-light fluorescence imaging system, in accordance with some examples. In a blue-light fluorescence imaging system, the excitation wavelength blocking filter may not be as effective as it overlaps the blue spectrum. Thus, the excitation light may leak into the image sensor (due to being in the visible range). Pulse width modulation of light source control, such as the examples described herein, can be particularly important for mitigating the image contamination. In some examples, white balancing may also be performed to further mitigate the image contamination.

### Ratio-Metric Segmentation of Fluorescence Data

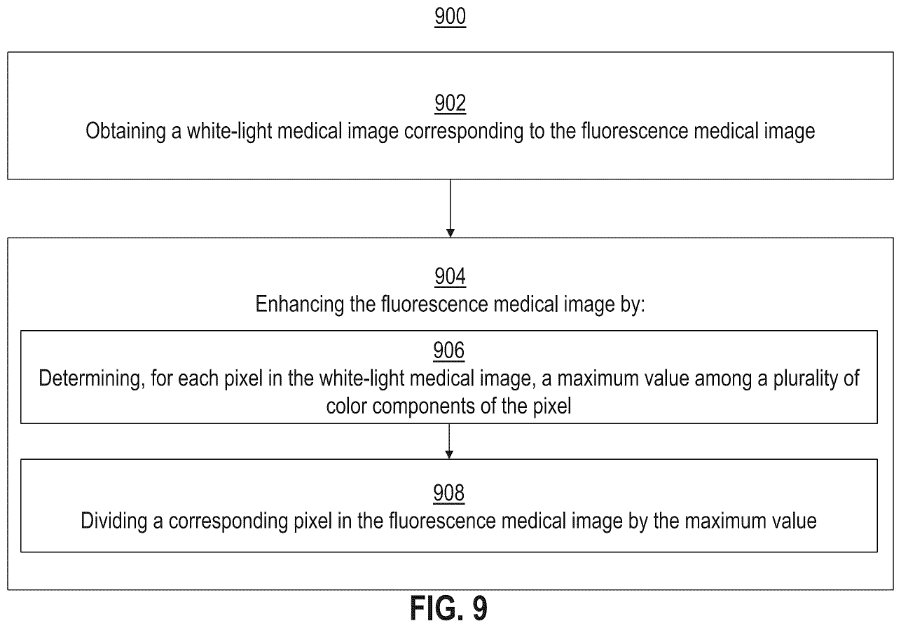

FIG. 9 illustrates an exemplary method 900 for enhancing a fluorescence medical image, according to some examples. Process 900 is performed, for example, using one or more electronic devices implementing a software platform. In some examples, process 400 is performed using a client-server system, and the blocks of process 900 are divided up in any manner between the server and one or more client devices. In some examples, process 900 is performed using only a client device or only multiple client devices. In process 900, some blocks are, optionally, combined, the order of some blocks is, optionally, changed, and some blocks are, optionally, omitted. In some examples, additional steps may be performed in combination with the process 900. Accordingly, the operations as illustrated (and described in greater detail below) are exemplary by nature and, as such, should not be viewed as limiting.

At block 902, an exemplary system receives a white-light medical image corresponding to the fluorescence medical image. The system may e.g. obtain the white-light medical image corresponding to the fluorescence medical image. In some examples, the fluorescence medical image and the white-light medical image depict the same tissue of a subject (e.g., a lymph node of the subject). In some examples, the two images are obtained using the same imager (e.g., a rolling shutter imager, a global shutter imager) that is part of an endoscopic imaging system or an open-field imaging system. At block 904, the system enhances the fluorescence medical image. The enhancement can comprise blocks 906 and 908. At block 906, the system determines, for each pixel in the white-light medical image, a maximum value among a plurality of color components of the pixel. At block 908, the system divides a corresponding pixel in the fluorescence medical image by the maximum value.

In some examples, the system displays the enhanced fluorescence medical image. In some examples, the enhanced fluorescence medical image is displayed according to a color scale in which different colors represent different fluorescence intensities. In some examples, according to the color scale, red indicates a higher fluorescence intensity than green and green indicates a higher fluorescence intensity than blue.

In some examples, displaying the enhanced fluorescence medical image comprises colorizing the corresponding white-light image based on the enhanced medical image and displaying the colorized white-light image. In some examples, the plurality of color components comprises: a red component, a green component, a blue component, or any combination thereof. In some examples, the enhanced fluorescence medical image is displayed as an overlay on the corresponding white-light image.

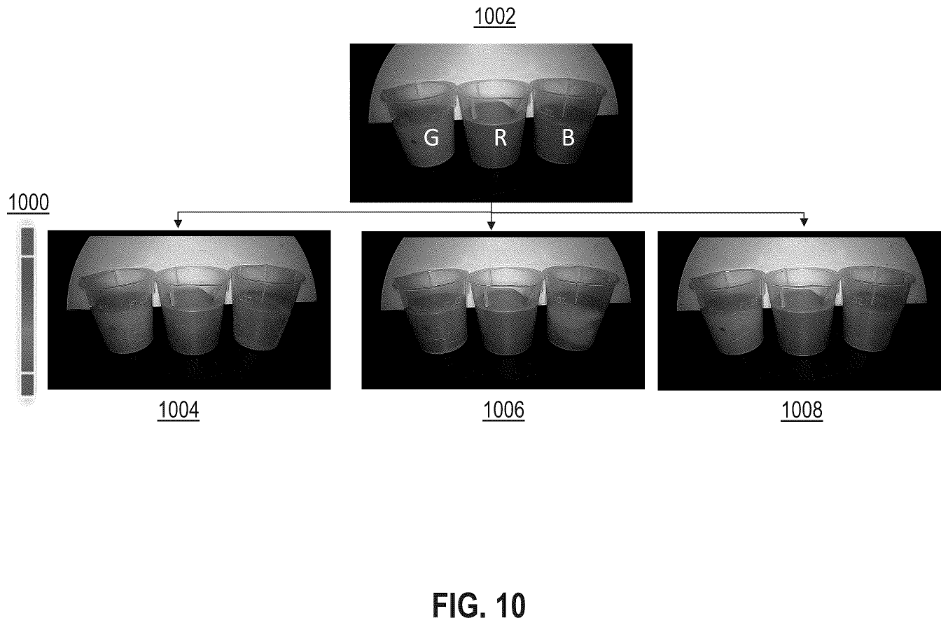

FIG. 10 illustrates a comparison between three segmented images 1004-1008, in accordance with some examples. Color segmentation can refer to the display of a fluorescence image according to a color scale in which different colors represent different fluorescence intensities. In the depicted example, according to the color scale 1000, red indicates a higher fluorescence intensity than green and green indicates a higher fluorescence intensity than blue.

Image 1002 is a white-light image of three cups containing a red-color liquid, a green-color liquid, and a blue-color liquid, respectively. Each of the liquids in the three cups is mixed with an equal concentration of a fluorescent dye, indocyanine green (ICG).

Image 1004 is a fluorescence image of the same three cups displayed according to the color scale 1000 without any normalization. In image 1004, the amplitude of each pixel is the ratio of the original fluorescence signal and a constant (i.e., IR/constant). No normalization is performed to the pixels.

Image 1006 is a fluorescence image of the same three cups displayed according to the color scale 1000 with normalization using red reflection. In image 1006, the amplitude of each pixel is the ratio of the original fluorescence signal and the red reflectance of a corresponding pixel in a corresponding white-light image (i.e., IR/R). As shown in FIG. 1006, the red reflectance (i.e., the R component) in blue is very low. Thus, the ratio of IR and R for the blue cup becomes very high, as indicated in the bright-red color of the blue cup in image 1006. The bright-red color incorrectly indicates a very high fluorescence signal even though the blue cup is known to contain the same concentration of ICG as the other cups, thus creating a false fluorescence signal which may compromise surgical safety.

Image 1008 is a fluorescence image of the same three cups displayed according to the color scale 1000 with normalization according to the process 900. Specifically, the system obtains a white-light medical image corresponding to the fluorescence medical image. For example, the white-light medical image may be the image frame captured immediately before the fluorescence image by the same imager. The system can then enhance the fluorescence medical image. First, the system can determine, for each pixel in the white-light medical image, a maximum value among a plurality of color components of the pixel, such as the red reflectance, the blue reflectance, and the green reflectance. The system can then divide each corresponding pixel in the fluorescence medical image by the maximum value (i.e., IR/max(RGB)). The enhanced image 1008 can then be displayed according to the color scale 1000. In image 1008, for the blue cup, because the blue component is dominating, the pixels corresponding to the blue cup are in fact divided by the blue component. Accordingly, the segmented image 1008 provides a more accurate representation of the fluorescence signals.

The foregoing description, for the purpose of explanation, has been described with reference to specific examples. However, the illustrative discussions above are not intended to be exhaustive or to limit the invention to the precise forms disclosed. For the purpose of clarity and a concise description, features are described herein as part of the same or separate examples; however, it will be appreciated that the scope of the disclosure includes examples having combinations of all or some of the features described. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

What is claimed is:

1. A method of imaging tissue of a subject using an imaging system comprising a rolling shutter imager, a fluorescence excitation illumination source, and a white-light illumination source, the method comprising:
illuminating the tissue of the subject with the white-light illumination source for a first illumination period to accumulate charge at a plurality of rows of pixels of the rolling shutter imager, wherein the fluorescence excitation illumination source is off during the first illumination period;
sequentially reading a first set of accumulated charge at the plurality of rows of pixels from a first row to a last row of the plurality of rows to produce a first set of imaging data;
illuminating the tissue of the subject with the fluorescence excitation illumination source for a second illumination period after the first illumination period to accumulate charge at the plurality of rows of pixels of the rolling shutter imager, wherein the white-light illumination source is off during the second illumination period;
sequentially reading a second set of accumulated charge at the plurality of rows of pixels from the first row to the last row of the plurality of rows to produce a second set of imaging data; and
generating one or more image frames based on the first set of imaging data and the second set of imaging data.

2. The method of claim 1, wherein the fluorescence excitation illumination source and the white-light illumination source are configured to alternately provide illumination periods to the tissue of the subject.

3. The method of claim 2, wherein the fluorescence excitation illumination source is configured to provide illumination periods at a first frequency and wherein the white-light illumination source is configured to provide illumination periods at a second frequency.

4. The method of claim 3, wherein the first frequency or the second frequency corresponds to the frame rate of the rolling shutter imager.

5. The method of claim 4, wherein the first frequency or the second frequency is half the frame rate of the rolling shutter imager.

6. The method of claim 1, wherein each illumination period of the white-light illumination source is a variable configured to not exceed a first maximum value.

7. The method of claim 6, wherein the first maximum value is about 1 millisecond.

8. The method of claim 1, wherein each illumination period of the fluorescence excitation illumination source is a fixed value.

9. The method of claim 1, wherein each illumination period of the fluorescence excitation illumination source is a variable configured to not exceed a second maximum value.

10. The method of claim 1, wherein the one or more image frames comprise a white-light image frame based on the first set of imaging data.

11. The method of claim 1, wherein the one or more image frames comprise a fluorescence image frame based on the second set of imaging data.

12. The method of claim 11, wherein the one or more image frames comprise a blended image frame based on the fluorescence image frame and the white-light image frame.

13. The method of claim 12, wherein the fluorescence image frame is overlaid on the white-light image frame in the blended image frame.

14. The method of claim 12, wherein the blended image frame is derived from colorizing the white-light image frame based on the fluorescence image frame.

15. The method of claim 1, further comprising: adding the one or more image frames to a video stream.

16. The method of claim 1, wherein the white-light illumination source comprises an LED.

17. The method of claim 1, wherein the fluorescence excitation illumination source comprises an infrared light, a blue-light illumination source, or any combination thereof.

18. The method of claim 1, wherein the rolling shutter imager is part of an endoscopic imager.

19. The method of claim 1, wherein the rolling shutter imager comprises a CMOS sensor.

20. A system of imaging tissue of a subject, the system comprising:
a fluorescence excitation illumination source,
a white-light illumination source, and
an imaging apparatus that comprises an electronic rolling shutter, the imaging apparatus being configured for:
illuminating the tissue of the subject with the white-light illumination source for a first illumination period to accumulate charge at a plurality of rows of pixels of the rolling shutter imager, wherein the fluorescence excitation illumination source is off during the first illumination period;
sequentially reading a first set of accumulated charge at the plurality of rows of pixels from a first row to a last row of the plurality of rows to produce a first set of imaging data;
illuminating the tissue of the subject with the fluorescence excitation illumination source for a second illumination period after the first illumination period to accumulate charge at the plurality of rows of pixels of the rolling shutter imager, wherein the white-light illumination source is off during the second illumination period;
sequentially reading a second set of accumulated charge at the plurality of rows of pixels from the first row to the last row of the plurality of rows to produce a second set of imaging data; and
generating one or more image frames based on the first set of imaging data and the second set of imaging data.
